(19) United States
(12) Patent Application Publication
(10) Pub. No.: US 2013/0274321 A1
(43) Pub. Date: Oct. 17, 2013

(54) Topical Compositions with Cannabis Extracts

(71) Applicant: Jon Newland, Palm Desert, CA (US)

(72) Inventor: Jon Newland, Palm Desert, CA (US)

(21) Appl. No.: 13/912,268

(22) Filed: Jun. 7, 2013

Related U.S. Application Data
(63) Continuation-in-part of application No. 13/434,927, filed on Mar. 30, 2012.

(51) Int. Cl. A61K 31/353

(52) U.S. Cl. CPC A61K 31/353
USPC 514/454

(57) ABSTRACT

The present invention provides a method for a topical treatment of a skin cyst. The method includes: applying a formulation to the skin cyst of a mammal; wherein the formulation includes: a dimethyl sulfoxide extract of mature, dried, powdered Cannabis sativa flower and bud leaves, wherein the mature, dried, powdered Cannabis sativa flower and bud leaves include greater than or equal to about 10 weight percent of tetrahydrocannibolinic acid and have been heated for approximately five minutes at a temperature greater than or equal to about 160° C. to less than about 193° C.

### RELATED APPLICATIONS

This application is a continuation-in-part of U.S. patent application Ser. No. 13/434,927 filed Mar. 30, 2012, which is hereby incorporated by reference in its entirety for all purposes.

### BACKGROUND OF THE INVENTION

Skin cysts are noncancerous, closed pockets of tissue that can be filled with fluid, pus, or other material. Skin cysts are common on the skin and can appear anywhere. They feel like large peas protruding from or under the surface of the skin. Cysts can develop as a result of infection, clogging of sebaceous glands (oil glands), or around foreign bodies, such as earrings.

Typically, cysts usually do not cause pain unless they rupture or become infected or inflamed. Most cysts do not disappear without treatment. Some cysts may need to be drained to relieve symptoms. That involves piercing the cyst with a sharp object and draining it. However, that doesn't cure the cyst. Some inflamed cysts can be treated with an injection of cortisone medication to cause it to shrink. Cysts that do not respond to other treatments or reoccur can be removed surgically if they cause troublesome symptoms.

What is needed is a topical composition for the treatment of skin cysts.

### SUMMARY OF THE INVENTION

The present invention provides a method for a topical treatment of a skin cyst. The method includes: applying a formulation to the skin cyst of a mammal; wherein the formulation includes: a dimethyl sulfoxide extract of mature, dried, powdered Cannabis sativa flower and bud leaves, and wherein the mature, dried, powdered Cannabis sativa flower and bud leaves include greater than or equal to about 10 weight percent of tetrahydrocannibolinic acid and have been heated for approximately five minutes at a temperature greater than or equal to about 160° C. to less than about 193° C.

In one embodiment, the mammal includes a human. In one embodiment, the formulation includes a solution, spray, lotion, gel, cream, or ointment. In one embodiment, the formulation includes a lotion.

In one embodiment, the skin cyst includes a sebaceous cyst, an epidermoid cyst, or a trichiemma cyst. In one embodiment, the skin cyst includes a sebaceous cyst. In one embodiment, the skin cyst includes an epidermoid cyst. In one embodiment, the skin cyst includes a trichiemma cyst.

The present invention provides a method for a topical treatment of a skin cyst. The method includes: applying a formulation to the skin cyst of a human; wherein the formulation includes: a dimethyl sulfoxide extract of mature, dried, powdered Cannabis sativa flower and bud leaves, and wherein the mature, dried, powdered Cannabis sativa flower and bud leaves include greater than or equal to about 10 weight percent of tetrahydrocannibolinic acid and have been heated for approximately five minutes at a temperature greater than or equal to about 160° C. to less than about 193° C. The present invention provides a method for a topical treatment of a skin cyst.

The method includes: applying a formulation to the skin cyst of a human; wherein the formulation consists essentially of a dimethyl sulfoxide extract of mature, dried, powdered Cannabis sativa flower and bud leaves, and wherein the mature, dried, powdered Cannabis sativa flower and bud leaves include greater than or equal to about 10 weight percent of tetrahydrocannibolinic acid and have been heated for approximately five minutes at a temperature greater than or equal to about 160° C. to less than about 193° C.

The present invention provides a method for a treatment of a skin cyst. The method includes: injecting a formulation into the skin cyst of a mammal; wherein the formulation comprises: a dimethyl sulfoxide extract of mature, dried, powdered Cannabis sativa flower and bud leaves, wherein the mature, dried, powdered Cannabis sativa flower and bud leaves comprise greater than or equal to about 10 weight percent of tetrahydrocannibolinic acid and have been heated for approximately five minutes at a temperature greater than or equal to about 160° C. to less than about 193° C.; allowing the formulation to be inside the skin cyst from about three minutes to about twenty four hours; and draining a fluid from the skin cyst.

In one embodiment, the method is performed two or more times. In one embodiment, the injecting is performed with a hypodermic needle. In one embodiment, the draining the fluid is performed by lancing, aspirating, or expressing the cyst.

### DETAILED DESCRIPTION OF THE INVENTION

The present invention provides a method for a topical treatment of a skin cyst. The method includes: applying a formulation to the skin cyst of a mammal; wherein the formulation includes: a dimethyl sulfoxide extract of mature, dried, powdered Cannabis sativa flower and bud leaves, wherein the mature, dried, powdered Cannabis sativa flower and bud leaves include greater than or equal to about 10 weight percent of tetrahydrocannibolinic acid and have been heated for approximately five minutes at a temperature greater than or equal to about 160° C.. to less than about 193° C..

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993, The American Heritage Dictionary of the English Language, Houghton Mifflin, Boston Mass., 1981, and Hawley's Condensed Chemical Dictionary, 14^{th} edition, Wiley Europe, 2002.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “abscess” refers to a collection of pus in any part of the body that, in most cases, causes swelling and inflammation around it.

As used herein, the term “agent” refers to anything that may have an impact on any living system such as a cell, nerve or tissue. For examples, the agent can be a chemical agent. The agent can also be a biological agent. The agent may include at least one known component. The agent can also be a physical agent.

As used herein, the term “administration” refers to a method of placing a device to a desired site. The placing of a device can be by any pharmaceutically accepted means such as by swallowing, retaining it within the mouth until the drug has been dispensed, placing it within the buccal cavity, inserting, implanting, attaching, etc. These and other methods of administration are known in the art.

As used herein, the term “aprotic solvent” refers to polar solvents of moderately high dielectric constant which do not contain acidic hydrogen. Examples of common aprotic solvents are dimethylsulfoxide (DMSO), dimethylformamide, sulfolane, tetrahydrofuran, diethyl ether, methyl-t-butyl ether, or 1,2-dimethoxyethane.

As used herein, the term “aqueous medium” refers to a liquid medium composed largely, but not necessarily exclusively, of water. Other components may also be present, such as salts, co-solvents, buffers, stabilizers, dispersants, colorants and the like.

As used herein, the term “biocompatible” refers to the material, substance, compound, molecule, polymer, or system, which does not cause severe toxicity, severe adverse biological reaction, or lethality in an animal when administered at reasonable doses and rates.

As used herein, the term “boil” refers to a skin infection involving an entire hair follicle and nearby skin tissue.

As used herein, the term “coating” refers to partial coating and adhesion or adsorption in addition to coating the whole surface of an object (e.g., core) which is to be coated.

As used herein, the term “cyst” refers to noncancerous, closed pockets of tissue that can be filled with fluid, pus, or other material.

As used herein, the term “an effective amount” refers to an amount sufficient to effect beneficial or desired results. An effective amount can be administered in one or more administrations, applications, or dosages. Determination of an effective amount for a given administration is well within the ordinary skill in the pharmaceutical arts.

As used herein, the terms “include,” “for example,” “such as,” and the like are used illustratively and are not intended to limit the present invention.

As used herein, the terms “individual,” “host,” “subject,” and “patient” are used interchangeably, and refer to a mammal, including, but not limited to, primates, including simians and humans.

As used herein, the term “infection” refers to the invasion of the host by germs that reproduce and multiply, causing disease by local cell injury, release of poisons, or germ-antibody reaction in the cells. The infection can be in a mammal (e.g., human).

As used herein, the term “inhibitor” refers to an agent that inhibits the growth of microbes.

As used herein, the term “liquid” refers to a substance that undergoes continuous deformation under a shearing stress. See, e.g., Concise Chemical and

Technical Dictionary, 4^{th} Edition, Chemical Publishing Co., Inc., p. 707, New York, N.Y. (1986).

As used herein, the term “mammal” refers to any of a class of warm-blooded higher vertebrates that nourish their young with milk secreted by mammary glands and have skin usually more or less covered with hair, and non- exclusively includes humans and non-human primates, their children, including neonates and adolescents, both male and female, livestock species, such as horses, cattle, sheep, and goats, and research and domestic species, including dogs, cats, mice, rats, guinea pigs, and rabbits.

As used herein, the term “MRSA” refers to Methicillin-Resistant Staphylococcus aureus. MRSA contains the SCCmec transposon. MRSA can be subtyped into type I, type II, type III, type IV or type IV.

As used herein, the term “type I MRSA” refers to MRSA that contains SCCmec type I. It is positive for nuc gene and mecA gene.

As used herein, the term “type II MRSA” refers to MRSA that contains SCCmec type II and is positive for nuc gene and mecA gene.

As used herein, the term “type III MRSA” refers to MRSA that contains SCCmec type III and is positive for nuc gene and mecA gene.

As used herein, the term “type IV MRSA” refers to MRSA that contains SCCmec type III and is positive for ccrAB gene, nuc gene and mecA gene.

As used herein, the term “HA-MRSA” refers to MRSA that contains SCCmec type I, II and III.

As used herein, the term “CA-MRSA” refers to MRSA that contains SCCmec type IV and is positive for PVL toxin.

As used herein, the terms “optional” or “optionally” mean that the subsequently described event or condition may need not occur, and that the description includes instances where the event or condition occurs and instances in which it does not.

As used herein, the term “patient” refers to a warm-blooded animal, and preferably a mammal, for example, a cat, dog, horse, cow, pig, mouse, rat, or primate, including a human.

As used herein, the term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. Several pharmaceutically acceptable ingredients are known in the art and official publications such as The United States Pharmacoepia describe the analytical criteria to assess the pharmaceutical acceptability of numerous ingredients of interest.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “prevent,” “preventative,” “prevention,” “protect,” and “protection” refer to medical procedures that keep the malcondition from occurring in the first place. The terms mean that there is no or a lessened development of disease or disorder where none had previously occurred, or no further disorder or disease development if there had already been development of the disorder or disease.

As used herein, the term “skin” refers to the external tissue layer in humans and animals consisting of epidermis and dermis.

As used herein, the term “epidermis” refers to the outer, protective, nonvascular layer of the skin of vertebrates, covering the dermis. The epidermis consists histologically of five layers, i.e. The stratum corneum, the stratum lucidum, the stratum granulosum, the stratum spinosum, and the stratum basale.

As used herein, the term “dermis” refers to the sensitive connective tissue layer of the skin located below the epidermis, containing nerve endings, sweat and sebaceous glands, and blood and lymph vessels. Histologically, the dermis consists of a papillary layer and a reticular layer. The papillary layer contains the vessels and nerve endings supplying the epidermis. The reticular consists predominantly of elastic fibers and collagen.

As used herein, the phrase “subcutaneous tissue layer” refers to a tissue layer located below the skin. This tissue layer is typically characterized by a loose meshwork of connective tissue such as collagen and elastic fibers. It is rich in small vessels, e.g., arterioles and venoles, and capillaries.

As used herein, the term “tissue” refers to an organized biomaterial usually composed of cells.

As used herein, the phrase “therapeutic kit” refers to a collection of components that can be used in a medical treatment.

As used herein, the terms “therapy,” and “therapeutic” refer to either “treatment” or “prevention,” thus, agents that either treat damage or prevent damage are “therapeutic.”

As used herein, the term “topically” refers to application of the compositions of the present invention to the surface of the skin and mucosal cells and tissues (e.g., alveolar, buccal, lingual, sublingual, masticatory, or nasal mucosa, and other tissues and cells which line hollow organs or body cavities).

As used herein, the terms “treating” or “treat” or “treatment” refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

As used herein, the term “treatment,” covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar, and “nm” denotes nanometer.

Concentrations, amounts, etc., of various components are often presented in a range format throughout this disclosure. The description in range format is merely for convenience and brevity and should not be construed as an inflexible limitation on the scope of the claimed invention. Accordingly, the description of a range should be considered to have specifically disclosed all the possible sub ranges as well as individual numerical values within that range. For example, description of a range such as 1% to 8% should be considered to have specifically disclosed sub ranges such as 1% to 7%, 2% to 8%, 2% to 6%, 3% to 6%, 4% to 8%, 3% to 8% etc., as well as individual numbers within that range, such as, 2%, 5%, 7% etc. This construction applies regardless of the breadth of the range and in all contexts throughout this disclosure.

The present invention provides a topical composition prepared by a process. The process includes heating mature, dried, powdered Cannabis sativa flower and bud leaves for about one minute to about ten minutes at a temperature greater than or equal to about 160° C.; and extracting the heated mature, dried, powdered Cannabis sativa flower and bud leaves with one or more aprotic solvents.

Preferably, the mature, dried, powdered Cannabis sativa flower and bud leaves include greater than or equal to about five weight percent of tetrahydrocannibolinic acid, more preferably, greater than or equal to about eight weight percent of tetrahydrocannibolinic acid, and most preferably, greater than or equal to about ten weight percent of tetrahydrocannibolinic acid.

Preferably, the mature, dried, powdered Cannabis sativa flower and bud leaves have been heated for about three minutes to about seven minutes at a temperature greater than or equal to about 160° C., more preferably, for about five minutes at a temperature greater than or equal to about 160° C.

Preferably, the mature, dried, powdered Cannabis sativa flower and bud leaves have been heated for about one minute to about ten minutes at a temperature less than about 193° C., more preferably, for about three minutes to about seven minutes at a temperature less than about 193° C., and most preferably, for about five minutes at a temperature less than about 193° C.

Preferably, the mature, dried, powdered Cannabis sativa flower and bud leaves have been heated for about one minute to about ten minutes at a temperature greater than or equal to about 160° C. to less than about 193° C., more preferably, for about three minutes to about seven minutes at a temperature greater than or equal to about 160° C. to less than about 193° C., and most preferably, for approximately five minutes at a temperature greater than or equal to about 160° C. to less than about 193° C. Preferably, the mature, dried, powdered Cannabis sativa flower and bud leaves include greater than or equal to about ten weight percent of tetrahydrocannibolinic acid and have been heated for approximately five minutes at a temperature greater than or equal to about 160° C. to less than about 193° C.

The topical composition may also include one or more chlorophyll extracts. Suitable chlorophyll extracts may include, for example, any of the commercial liquid chlorophyll extracts supplied by Spectrum Chemical Manufacturing Corporation (Gardena, Calif., US). Chlorophyll extracts appear to reduce the garlic taste that many people develop after dimethylsulfoxide is applied to the skin or ingested. Typically, chlorophyll extracts, if present, are present in a total amount by weight of about 0.25%, about 0.5%, about 0.75%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, or about 5.0%.

Preferably, the one or more aprotic solvents include acetonitrile, dimethyl formamide, dimethyl sulfoxide, or combinations thereof, and more preferably, the one or more aprotic solvents include dimethyl sulfoxide. Preferably, the dimethyl sulfoxide is pharmaceutical grade.

Preferably, the extraction is performed from about 0° C. to about 100° C., more preferably, from about 15° C. to about 40° C., and most preferably, at about room temperature.

Preferably, the extraction is performed for about 1 hour to about 72 hours, more preferably, for about 12 hours to about 36 hours, and most preferably, for about 24 hours.

Typically, the topical composition is filtered after extraction through a sintered glass filter, a plastic screen, or filter paper.

The topical composition may be used in many forms, preferably, a solution, spray, lotion, gel, cream, or ointment, and more preferably as a lotion.

The topical composition may also include, for example, one or more solvents, one or more thickening agents, one or more penetration enhancers, one or more wetting agents, one or more lubricants, one or more emollients, one or more fragrances, one or more pigments, or a combination thereof.

Typically, the topical composition is used to treat, for example, pain, inflammation, muscle tightness, muscle spasms, skin ulcerations, and scleroderma. Preferably, the topical composition is used to treat joint pain, muscle pain, or arthritis. Typically, the topical composition is used to treat, for example, post-herpetic neuralgia, shingles, burns, actinic keratosis, oral cavity sores, oral ulcers, post-episiotomy pain, psoriasis, pruritis, contact dermatitis, eczema, bullous dermatitis herpetiformis, exfoliative dermatitis, mycosis fungoides, pemphigus, severe erythema multiforme, seborrheic dermatitis, psoriatic arthritis, diabetic neuropathy, ankylosing spondylitis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, juvenile rheumatoid arthritis, neurodermitis, contact eczema, allergies, phototoxic reactions, inflammatory and itching dermatoses, rosacea, perioral dermatitis, acne, acne conglobata, psoriasis, mosquito bites, skin atrophy, allergic rhinitis, privinismus, conjunctivitis, otitis externa, bronchial asthma, Crohn's disease, ulcerative colitis, sarcoidosis, inflammatory-rheumatic diseases of the soft tissue or joints, mycoses, or combinations thereof.

Various methods for the topical treatment of pain, inflammation, muscle tightness, muscle spasms, skin ulcerations, and scleroderma are also provided. These methods include applying a topical composition directly to the skin or by use of a patch, bandage, and the like. The topical composition may be used at about room temperature, heated above room temperature prior to applying it to the skin, or cooled prior to applying it to the skin.

Various methods of making a topical composition are also provided.

Various kits are also provided. Typically, the kits include a topical composition and instructions for the use of the topical composition and dosage regime thereto.

The topical compositions, as described herein, may also include one or more optional ingredients, for example, palliative agents, skin conditioning agents, emollients, humectants, odorants, preservatives, solvents, thickening, stiffening and suspending agents, other agents, or a combination thereof.

Typically, the one or more optional ingredients, if present, are present in an amount of about 0.001% to about 30%, about 3% to about 25%, or about 5% to about 15%, by weight. Illustratively, one or more emollients are present in a total amount of about 0.001%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight percent.

Suitable palliative agents include, for example, menthol, camphor, phenol, allantoin, benzocaine, corticosteroids, phenol, zinc oxide, camphor, pramoxine, dimethicone, meradimate, octinoxate, octisalate, oxybenzone, dyclonine, benzyl alcohol, mineral oil, propylene glycol, titanium dioxide, magnesium stearate, and the like, or a combination thereof.

Suitable skin conditioning agents include, for example, mineral oil, petrolatum, dimethicone, dimethicone copolyol, cationic monomers and polymers (such as guar hydroxypropyl trimonium chloride and distearyl dimethyl ammonium chloride), and combinations thereof Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose, and combinations thereof.

Suitable emollients include, for example, caprylic/capric triglycerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, gyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, urea, and combinations thereof.

Suitable humectants include, for example, glycerine, propylene glycol, sorbitol, urea, and combinations thereof.

Suitable odorants include, for example, hypoallergenic perfume, menthol, and combinations thereof.

Suitable preservatives, antioxidants, and chemical stabilizers include, for example, alcohol, benzyl alcohol, butylated hydroxyanisole, butylparaben, calcium acetate, castor oil, chlorocresol, 4-chloro-m-cresol, citric acid, disodium edetate, edetate disodium, ethoxylated alcohol, ethyl alcohol, glycerin, methylparaben, parabens, potassium sorbate, propyl gallate, propylene glycol, propylparaben, sodium bisulfate, sodium citrate, sodium metabisulfite, sorbic acid, tannic acid, triglycerides of saturated fatty acids, zinc stearate, and combinations thereof.

Suitable solvents include, for example, alcohol, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palmitate, mineral oil, phosphoric acid, polyethylene glycol 300, polyethylene glycol 400, polyethylene glycol 1450, polyethylene glycol 8000, polyethylene glycol 1000 monocetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, propylene carbonate, propylene glycol, purified water, and SD alcohol 40, triglycerides of saturated fatty acids, and combinations thereof.

For patients with sensitive skin, the topical compositions, as described herein, may be diluted with purified water. For example, topical compositions may be formulated from about 1 weight percent to about 99 weight percent water.

Suitable thickening, stiffening and suspending agents include, for example, aluminum stearate, beeswax, synthetic beeswax, carbomer 934, carbomer 934P, carbomer 940, cetostearyl alcohol, cetyl alcohol, cetyl esters wax, dextrin, glyceryl monostearate, hydroxypropyl cellulose, kaolin, paraffin, petrolatum, polyethylene, propylene glycol stearate, starch, stearyl alcohol, wax, white wax, xanthan gum, bentonite, and combinations thereof.

Other optional agents may be added to the composition including, for example, aloe, arachis oil, benzoic acid, cocoa butter, coenzyme Q10, Q10, dimethicone, eucalyptus oil, resorcinol, retinol, retinyl palmitate, retinyl acetate, fennel extract, whey protein, ceramide, silicone, alpha-hydroxy acids, beta-hydroxy acids, sorbitol, vitamin A, vitamin B, vitamin C, vitamin D, vitamin E, and vitamin K. Unless otherwise indicated, the composition will generally contain less than about 5% by weight and typically less than about 1% by weight of the above-ingredients.

The topical compositions, as described herein, may be applied in a single administration or in multiple administrations. The compositions are topically applied for at least one day, at least two days, at least three days, at least four days, at least 5 days, once a week, at least twice a week, at least once a day, at least twice a day, multiple times daily, multiple times weekly, biweekly, at least once a month, or any combination thereof.

The topical compositions, as described herein, may be topically applied for a period of time of about one month, about two months, about three months, about four months, about five months, about six months, about seven months, about eight months, about nine months, about ten months, about eleven months, about one year, about 1.5 years, about 2years, about 2.5 years, about 3 years, about 3.5 years, about 4 years, about 4.5 years, and about 5 years.

Preferably, the composition is applied topically to the area of pain until the pain subsides. The composition is preferably administered six to eight times a day for from one day to a week or more until healing occurs.

Dosage forms of topical compositions, as described herein, include, for example, patches, ointments, creams, emulsions, liquids, lotions, gels, bioadhesive gels, aerosols, shampoos, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within the epidermis, the dermis and keratin layers. Preferably, the topical composition, as described herein, is a liquid that can be easily applied to the area of pain.

The topical compositions, as described herein, can be applied to any bodily region needing treatment, including, for example, the oral facial region, the eye, the uro-genital region (external or internal, skin or mucosa), vaginal mucosa, rectal mucosa, anal mucosa, oral mucosa, extremities, skin, oral pharynx, superficial skin structure and appendages, lips, vermillion border, mouth, neck, perineum, upper legs, hand, cornea, eye, urethra, or a combination thereof.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention.

Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The invention should now be illustrated with the following non-limiting examples.

### EXAMPLES

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Example 1

### Preparation of a Topical Composition

To an open vessel was added about 200 grams of mature, dried, powdered Cannabis sativa flower and bud leaves, which included greater than or equal to about 10 weight percent of tetrahydrocannibolinic acid. The vessel was heated for approximately five minutes at a temperature greater than or equal to about 160° C. to less than about 193° C. and cooled to about room temperature.

About 120 grams of the heat-treated Cannabis sativa flower and bud leaves was ground up into a fine powder. The ground up powder was added to one liter of pharmaceutical grade dimethyl sulfoxide. The mixture was stirred for about 24 hours at about room temperature. The mixture was filtered to afford the topical composition.

### Example 2

### Application of a Topical Composition

To a painful joint of a human was applied the topical composition prepared in Example 1 with a spray atomizer. Within a few minutes, the pain had subsided.

### Example 3

### Preparation of a Patch with a Topical Composition

To a backing layer including a patch is placed an effective amount of the topical composition prepared in Example 1.

### Example 4

### Application of a Topical Composition in a Patch

To a painful joint of a human is applied the patch from Example 3 so that the topical composition will contact with the skin. Within a few minutes, the pain subsides.

### Example 5

### Preparation of a Strip with a Topical Composition

To a backing layer including a strip is placed an effective amount of the topical composition prepared in Example 1.

### Example 6

### Application of a Topical Composition in a Strip

To a painful joint of a human is applied the strip from Example 5 so that the topical composition will contact with the skin. Within a few minutes, the pain subsides.

### Example 7

### Preparation of a Bandage with a Topical Composition

To a backing layer including a bandage is placed an effective amount of the topical composition prepared in Example 1.

### Example 8

### Application of a Topical Composition in a Bandage

To a painful joint of a human is applied the bandage from Example 7 so that the topical composition will contact with the skin. Within a few minutes, the pain subsides.

### Example 9

### Preparation of a Covering with a Topical Composition

To a backing layer including a covering is placed an effective amount of the topical composition prepared in Example 1.

### Example 10

### Application of a Topical Composition in a Covering

To a painful joint of a human is applied the covering from Example 9 so that the topical composition will contact with the skin. Within a few minutes, the pain subsides.

### Example 11

### Application of a Topical Composition

A middle-aged woman with multiple methicillin-resistant Staphylococcus aureus (MRSA)-based boil infections, which typically develop into deep abscesses if left untreated and require lancing and oral antibiotics to manage, applied the topical composition prepared in Example 1. Upon repeated applications, these boil infections did not develop into deep abscesses.

### Example 12

### Application of a Topical Composition

An 18 year-old male man with multiple methicillin-resistant Staphylococcus aureus (MRSA)-based boil infections, which typically develop into deep abscesses if left untreated and require lancing and oral antibiotics to manage, applied the topical composition prepared in Example 1. Upon repeated applications, these boil infections cleared up.

### Example 13

### Application of a Topical Composition

A middle-aged woman with boil infections, which did not clear up with an over-the-counter antibiotic cream, applied the topical composition prepared in Example 1. These boil infections cleared up within days.

### Example 14

### Application of a Topical Composition

A middle-aged man suffering from a recurrence of a surgically excised cyst on the neck applied the topical composition prepared in Example 1 daily for several weeks. The cyst disappeared completely.

### Example 15

### Treatment of a Cyst

A patient with recurring pilonidal disease with a cyst, which is abscessed is treated by incising and draining the cyst, followed by irrigation (or injection) with a 70% percent DMSO extract of cannabis, as prepared in Example 1, into the sinus of the cyst and injection into the pits, or openings, of the fistula or fistulas feeding the cyst. This procedure is repeated until the cyst is reabsorbed and pits close.

### Example 16

### Treatment of a Cyst

A patient with recurring pilonidal disease with a cyst, which is not abscessed is treated by injection with a 70% percent DMSO extract of cannabis, as prepared in Example 1, into the sinus of the cyst and injection into the pits, or openings, of the fistula or fistulas feeding the cyst. This procedure is repeated until the cyst is reabsorbed and pits close.

### Example 16

### Treatment of a Cyst

A patient with a recurring sebaceous cyst or other cyst, which is abscessed is treated by incising and draining the cyst, followed by irrigation (or injection) with a 70% percent DMSO extract of cannabis, as prepared in Example 1, into the sinus of the cyst and injection into the pits, or openings, of the fistula or fistulas feeding the cyst. This procedure is repeated until the cyst is reabsorbed and the fistula or fistulas close.

### Example 18

### Treatment of a Cyst

A patient with a recurring sebaceous cyst or other cyst, which is not abscessed, is treated by injection with a 70% percent DMSO extract of cannabis, as prepared in Example 1, into the sinus of the cyst and injection into the pits, or openings, of the fistula or fistulas feeding the cyst. This procedure is repeated until the cyst is reabsorbed and the fistula or fistulas close.

What is claimed is:

1. A method for a topical treatment of a skin cyst comprising:
applying a formulation to the skin cyst of a mammal; wherein the formulation comprises:
a dimethyl sulfoxide extract of mature, dried, powdered Cannabis sativa flower and bud leaves, and
wherein the mature, dried, powdered Cannabis sativa flower and bud leaves comprise greater than or equal to about 10 weight percent of tetrahydrocannibolinic acid and have been heated for approximately five minutes at a temperature greater than or equal to about 160° C. to less than about 193° C.

2. The method of claim 1, wherein the mammal comprises a human.

3. The method of claim 1, wherein the formulation comprises a solution, a spray, a lotion, a gel, a cream, or an ointment.

4. The method of claim 3, wherein the formulation comprises the lotion.

5. The method of claim 1, wherein the skin cyst comprises a sebaceous cyst, an epidermoid cyst, or a trichiemma cyst.

6. The method of claim 5, wherein the skin cyst comprises the sebaceous cyst.

7. The method of claim 5, wherein the skin cyst comprises the epidermoid cyst.

8. The method of claim 5, wherein the skin cyst comprises the trichiemma cyst.

9. A method for a topical treatment of a skin cyst comprising:
applying a formulation to the skin cyst of a human; wherein the formulation comprises:
a dimethyl sulfoxide extract of mature, dried, powdered Cannabis sativa flower and bud leaves, and
wherein the mature, dried, powdered Cannabis sativa flower and bud leaves comprise greater than or equal to about 10 weight percent of tetrahydrocannibolinic acid and have been heated for approximately five minutes at a temperature greater than or equal to about 160° C. to less than about 193° C.

10. The method of claim 9, wherein the formulation comprises a solution, a spray, a lotion, a gel, a cream, or an ointment.

11. The method of claim 10, wherein the formulation comprises the lotion.

12. The method of claim 9, wherein the skin cyst comprises a sebaceous cyst, an epidermoid cyst, or a trichiemma cyst.

13. The method of claim 12, wherein the skin cyst comprises the sebaceous cyst.

14. The method of claim 12, wherein the skin cyst comprises the epidermoid cyst.

15. The method of claim 12, wherein the skin cyst comprises the trichiemma cyst.

16. A method for a treatment of a skin cyst comprising:
injecting a formulation into the skin cyst of a mammal; wherein the formulation comprises:
a dimethyl sulfoxide extract of mature, dried, powdered Cannabis sativa flower and bud leaves,
wherein the mature, dried, powdered Cannabis sativa flower and bud leaves comprise greater than or equal to about 10 weight percent of tetrahydrocannibolinic acid and have been heated for approximately five minutes at a temperature greater than or equal to about 160° C. to less than about 193° C.;
allowing the formulation to be inside the skin cyst from about three minutes to about twenty four hours; and
draining a fluid from the skin cyst.

17. The method of claim 16, wherein the skin cyst comprises a sebaceous cyst, an epidermoid cyst, or a trichiemma cyst.

18. The method of claim 17, wherein the method is performed two or more times.

19. The method of claim 17, wherein the injecting is performed with a hypodermic needle.

20. The method of claim 17, wherein the draining the fluid is performed by lancing, aspirating, or expressing the cyst.
